(19) United States
(12) Patent Application Publication
(10) Pub. No.: US 2007/0041910 A1
(43) Pub. Date: Feb. 22, 2007

(54) Pharmaceutical spray compositions comprising a bioactive agent, at least one volatile silicone and a non-volatile oily phase

(75) Inventors: Franck Pitre, Bussy Saint Georges (FR); Laurent Fredon, Roquefort Les Pins (FR); Claire Mallard, Mougins Le Haut (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/432,410

(22) Filed: May 12, 2006

Related U.S. Application Data
(63) Continuation of application No. PCT/EP04/14085, filed on Nov. 17, 2004.

(30) Foreign Application Priority Data
Nov. 21, 2003 (FR) 03/13660

(51) Int. Cl. A61L 9/04; A61K 31/59

(52) U.S. Cl. 424/45; 514/167

(57) ABSTRACT

Sprayable pharmaceutical compositions suited for improved transdermal penetration contain a) at least one bioactive agent, notably at least one derivative of vitamin D, (b) at least one volatile silicone and c) a non-volatile oily phase, formulated into d) a pharmaceutically acceptable vehicle therefor.

### CROSS-REFERENCE TO PRIORITY/PCT/PROVISIONAL APPLICATIONS

This application claims priority under 35 U.S.C. § 119 of FR 03/13660, filed Nov. 21, 2003, and of provisional application Ser. No. 60/541,245, filed Feb. 4, 2004, and is a continuation of PCT/EP 2004/014085 filed Nov. 17, 2004 and designating the United States, published in the English language as WO 2005/053666 A1 on Jun. 16, 2005, each hereby expressly incorporated by reference and each assigned to the assignee hereof.

### BACKGROUND OF THE INVENTION

1. Technical Field of the Invention

The present invention relates to compositions comprising a pharmaceutical active agent, at least one volatile silicone and a non-volatile oily phase, formulated into a physiologically acceptable medium, to the process for preparing same and to applications thereof in cosmetics and in dermatology. The subject compositions make it possible to obtain good penetration of the active agent through the layers of the skin.

2. Description of Background and/or Related and/or Prior Art

In the field of dermatology and of the formulation of pharmaceutical compositions, those skilled in this art seek compositions which make it possible to release the active agent and to promote its penetration through the layers of the skin in order to improve its effectiveness. Such products should also exhibit good cosmeticity and preferably be nonirritant.

There currently exist many topical compositions comprising an active agent and making it possible to promote penetration thereof into the skin by means of the presence, in particular, of a high content of pro-penetrating glycol. These compositions are formulated in the form of emulsions with a high content of fatty phase, which are commonly called “lipocreams”, in the form of anhydrous compositions which are called “ointments”, in the form of fluid compositions with a high content of volatile solvents, such as ethanol or isopropanol, intended for application to the scalp, also called “hair lotions”, or else in the form of viscous O/W emulsions, which are also called “O/W creams”.

O/W creams comprising a corticoid and a high percentage of propylene glycol (47.5%), marketed under the trademark TEMOVATE® by GLAXOSMITHKLINE, are, for example, known. The stabilizing of a formulation comprising such a percentage of glycol makes it necessary to include, in the emulsion, emulsifiers and stabilizers of the glyceryl stearate or PEG 100 stearate type, or alternatively stabilizers or consistency factors of the white wax or cetostearyl alcohol type, which result in the formation of a viscous cream, namely, a cream with a viscosity greater than 10 Pa·s (10,000 centipoises, measured with a Brookfield model LVDV II+mobile No. 4 device, at a rate of 30 rpm for 30 seconds and at a temperature of 25° C.±3° C.). This viscosity therefore makes the product difficult to apply. These compositions therefore show, firstly, poor cosmetic acceptability due to their viscosity and, secondly, risks of intolerance caused by the presence of high proportions of glycol. Those skilled in the art therefore seek to improve these parameters.

In order to facilitate the application of topical compositions comprising a high percentage of pro-penetrating glycol, the assignee hereof has developed, described in EP-832,647, a lotion, which is a stable formulation of O/W emulsion type, and the viscosity of which is intermediate from hair lotions which are too fluid and have too limited a use, and O/W creams which are too viscous and have a greasy and sticky side to them, while at the same time conserving the pro-penetrating properties of the glycol. These formulae effectively show good penetration of the active agent, but still comprise a high percentage of glycol which can therefore induce a sticky effect or problems of tolerance resulting in moderate acceptability of the product by the patient.

Formulations containing silicone compounds which result in compositions which are pleasant to use are, moreover, known to those skilled in the art. Thus, in U.S. Pat. No. 6,538,039, a novel formulation of active agent for transdermal administration has been developed, comprising silicone compounds in order to deposit a film at the surface of the skin. In that application also, the transdermal passage is facilitated by the obligatory presence of absorption promoters, namely, among other compounds mentioned, glycols.

In EP-0-966,972, the compositions described can be formulated in the form of a spray and comprise an active compound, a silicone gum and a pharmaceutically acceptable excipient. The problem that the invention described in EP-0-966,972 proposes to solve is that of depositing a substantive film at the surface of the skin, which problem is solved by means of the presence of the silicone gum.

The disadvantage or drawback that the present invention here proposes to solve is that of designing a composition for improving the penetration of the pharmaceutical active agent, and its rapidity of penetration over time, in order to improve its therapeutic efficacy, while at the same time avoiding the presence of a high content of glycol. The compositions according to the invention should also be easy to use and exhibit a cosmeticity which is acceptable for application to all of the regions of the body which may be affected by the pathology.

EP-0-966,972 and U.S. Pat. No. 6,538,039 represent the prior art most akin to the present invention, given the composition of the formulations described. However, nothing in this prior art would suggest to those skilled in the art compositions according to the invention in order to obtain good penetration of the active agent incorporated, into the layers of the skin.

### SUMMARY OF THE INVENTION

Thus, it has now surprisingly been found that compositions comprising, formulated into a pharmaceutically acceptable vehicle:
a) a therapeutically effective amount of a pharmaceutical bioactive agent,
b) at least one volatile silicone,
c) a non-volatile oily phase,
   provide an improvement in penetration of the active agent.

The compositions of the present invention, while allowing good penetration of the active principles, also exhibit very good acceptability and tolerance among patients, as described in Examples 8 and 9 to follow. It is therefore found that the compositions according to the invention are particularly suitable for the treatment of dermatological conditions and afflictions, and more particularly very suitable for the treatment of psoriasis.

### DETAILED DESCRIPTION OF BEST MODE AND SPECIFIC/PREFERRED EMBODIMENTS OF THE INVENTION

The present invention relates more particularly to compositions comprising, formulated into a pharmaceutically acceptable vehicle:
a) a therapeutically effective amount of a pharmaceutical bioactive agent,
b) at least one volatile silicone,
c) a non-volatile oily phase,
   wherein the pharmaceutical bioactive agent is a compound derived from vitamin D.

The term “compound derived from vitamin D” means compounds which exhibit biological properties similar to those of vitamin D, in particular the properties of trans-activation of vitamin D response elements (VDREs), such as agonist or antagonist activity with respect to receptors for vitamin D or for derivatives thereof. Compounds derived from vitamin D that are useful according to the invention thus comprise structural analogues, for example bioaromatic analogues. The expression “vitamins D or their derivatives” means, for example, the derivatives of vitamin D₂ or D₃, and in particular 1,25-dihydroxy vitamin D₃ (calcitriol).

Among the pharmaceutical bioactive agents derived from vitamin D which can be used according to the invention, mention may be made, by way of non-limiting examples, of the compounds described in EP-1-124,779, EP-1-235,824, EP-1,235,777, WO 02/94754 and WO 03/050067.

Preferably, the vitamin D derivatives according to the invention are the compounds described in FR-2-785,284, incorporated herein by way of reference. These are compounds which are structural analogues of vitamin D and which exhibit selective activity on cell proliferation and differentiation without being hypercalcian-inducing in nature.

These compounds can be represented by general formula (I) below: 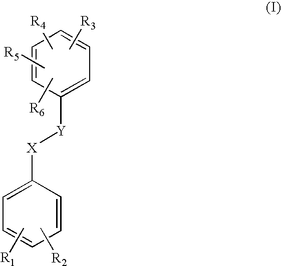
in which:

R₁ is a hydrogen atom, a methyl radical or a radical —(CH₂)ₙ—OR₇;

R₂ is a radical —(CH₂)ₙ—OR₈;

n, R₇ and R₈ are as defined below;

X—Y is a bond selected from the bonds of formulae (a) to (d) below which can be read from left to right or vice versa:

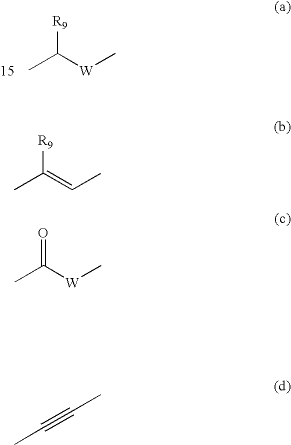

R₉ and W having the meanings given below;

R₃ is the chain of vitamin D₂ or vitamin D₃; 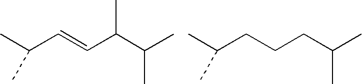
the dashed lines represent the bond linking the chain to the benzene ring represented in formula (I),
or R₃ is a chain having from 4 to 8 carbon atoms substituted with one or more hydroxyl groups, with the proviso that the hydroxyl groups may be protected in acetoxy, methoxy or ethoxy, trimethylsilyloxy, tert-butyldimethylsilyloxy or tetrahydropyranyloxy form, and optionally, in addition:

substituted with one or more lower alkyl or cycloalkyl groups and/or

substituted with one or more halogen atoms and/or

substituted with one or more groups CF₃ and/or

in which one or more carbon atoms of the chain are replaced with one or more oxygen, sulfur or nitrogen atoms, with the proviso that the nitrogen atoms may be optionally substituted with lower alkyl radicals and/or

in which one or more single bonds of the chain are replaced with one or more double and/or triple bonds,

R₃ being positioned, on the benzene ring, in the position para- or meta- to the X—Y bond;

R₄, R₅ and R₆, two of which may be identical or different, are each a hydrogen atom, a lower alkyl radical, a halogen atom, a radical —OR₁₀ or a polyether radical;

R₁₀ is as defined below;

n is 0, 1 or 2;

R₇ and R₈, which may be identical or different, are each a hydrogen atom, an acetyl radical, a trimethylsilyl radical, a tert-butyidimethylsilyl radical or a tetrahydropyranyl radical;

R₉ is a hydrogen atom or a lower alkyl radical;

W is an oxygen or sulfur atom, a radical —CH₂— or a radical —NH— which can optionally be substituted with a lower alkyl radical;

R₁₀ is a hydrogen atom or a lower alkyl radical;

and also the optical and geometrical isomers of said compounds of formula (I), and the salts thereof when X—Y are each a bond of formula (a) and W is a radical —NH— optionally substituted with a lower alkyl radical.

Among the compounds of formula (I) in the compositions of the present invention, mention may in particular be made of the following:

1. 6-[3-(3,4-bishydroxymethylbenzyloxy)phenyl]-2-methylhepta-3,5-dien-2-ol,

2. 7-[3-(3,4-bishydroxymethylphenoxymethyl)phenyl]-3-ethyloctan-3-ol,

3. 7-{3-[2-(3,4-bishydroxymethylphenyl)ethyl]phenyl}-3-ethylocta-4,6-dien-3-ol,

4. 6-{3-[2-(3,4-bishydroxymethylphenyl)ethyl]phenyl}-2-methylhepta-3,5-dien-2-ol,

5. 7-{3-[2-(3,4-bishydroxymethylphenyl)vinyl]phenyl}-3-ethylocta-4,6-dien-3-ol,

6. 7-[3-(3,4-bishydroxymethylbenzyloxy)phenyl]-3-ethyl-3-octanol,

7. 4E,6E)-7-[3-(3,4-bishydroxymethylbenzyloxy)phenyl]-3-ethylocta-4,6-dien-3-ol,

8. (4E,6E)-7-[3-(3,4-bishydroxymethylbenzyloxy)phenyl]-3-ethylnona-4,6-dien-3-ol,

9. (E)-7-[3-(3,4-bishydroxymethylbenzyloxy)phenyl]-3-ethyloct-4-en-3-ol,

10. (E)-7-[3-(3,4-bishydroxymethylbenzyloxy)phenyl]-3-ethyloct-6-en-3-ol,

11. (E)-7-[3-(3,4-bishydroxymethylbenzyloxy)phenyl]-3-ethyloct-6-en-4-yn-3-ol,

12. (4E,6E)-7-[3-(3,4-bishydroxymethylphenoxymethyl)phenyl]-3-ethylocta-4,6-dien-3-ol,

13. (E)-7-[3-(3,4-bishydroxymethylphenoxymethyl)phenyl]-3-ethylnon-6-en-3-ol,

14. (E)-7-{3-[(3,4-bishydroxymethylbenzyl)methylamino]phenyl}-3-ethyloct-6-en-3-ol, and

15. 7-[3-(3,4-bishydroxymethylbenzyloxy)phenyl]-3-ethyl-7-methyloctan-3-ol.

More preferably, the pharmaceutical active agent incorporated into the compositions according to the invention is (4E,6E)-7-[3-(3,4-bishydroxymethylbenzyloxy)phenyl]-3-ethylnona-4,6-dien-3-ol.

Advantageously, the compositions according to the invention comprise from 0.0001 to 20% by weight, relative to the total weight of the composition, of an active agent, preferably from 0.025 to 15% by weight, and more preferably from 0.01 to 5% by weight.

Of course, the amount of active agent in the composition according to the invention will depend on the active agent under consideration.

The compositions according to the invention will preferably comprise an active agent derived from vitamin D at a concentration of less than 2% by weight of active agent, preferably of from 0.025 to 0.5% by weight. The preferred pharmaceutical active agent according to the invention is (4E,6E)-7-[3-(3,4-bishydroxymethylbenzyloxy)phenyl]-3-ethylnona-4,6-dien-3-ol used at a concentration of 0.3% by weight.

The active agents according to the invention may be used alone or in combination.

According to the invention, the term “volatile silicone” means polyorganosiloxane compounds, which may be cyclic or linear, having a measurable pressure under ambient conditions. The cyclic volatile silicones according to the invention are polydimethylcyclosiloxanes, i.e., compounds of formula: 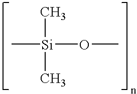
with n being, on average, from 3 and 6, and preferably n=4 or n=5, generally known as cyclomethicones. The linear volatile silicones according to the invention are linear polysiloxanes such as hexamethyldisiloxane or low molecular weight dimethicones. The linear volatile silicones generally have a viscosity of less than approximately 5 centistokes at 25° Celsius, whereas the cyclic volatile silicones have a viscosity of less than approximately 10 centistokes at 25° Celsius.

Preferred volatile silicones according to the invention are the linear siloxanes, and more preferably hexamethyldisiloxane. By way of example, mention may be made of the product marketed by DOW CORNING, DC Fluid 0.65cSt.

Advantageously, the compositions according to the invention comprise from 25 to 95% by weight, relative to the total weight of the composition, of the volatile silicone, and preferably from 40 to 80% by weight, and more preferably from 55 to 65% by weight.

According to the invention, the term “non-volatile oily phase” means a variety of non-volatile oil suitable for a pharmaceutical or cosmetic composition. The non-volatile oils generally have a viscosity of greater than approximately 10 centipoises at 25° C., and can attain a viscosity ranging up to 1,000,000 centipoises at 25° C. The non-volatile oily phase can be constituted of a large variety of synthetic or natural, silicone or organic oils, a non-exhaustive list of which is given by way of illustration:

(a) Esters:

Examples of a non-volatile oil according to the invention comprise esters of formula RCO—OR′ with R and R′, which may be identical or different, representing a linear or branched chain of an alkyl, alkenyl, alkoxycarbonylalkyl or alkoxycarbonyloxyalkyl radical having from 1 to 25 carbon atoms, preferably from 4 to 20 carbon atoms. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco dicaprylate/caprate, decyl isostearate, isodecyl oleate, isodecyl neopentanoate, isohexyl neopentanoate, octyl palmitate, dioctyl malate, tridecyl octanoate, myristyl myristate and octododecanol.

(b) Glyceryl Esters of Fatty Acids:

The oil may also comprise fatty esters of natural fatty acids, or triglycerides of animal or plant origin. Such examples include, castor oil, lanolin oil, triisocetyl citrate, triglycerides having from 10 to 18 carbon atoms, caprylic/capric triglycerides, coconut oil, corn oil, cottonseed oil, flax oil, mink oil, olive oil, palm oil, illipe butter, rapeseed oil, soybean oil, sunflower oil, nut oil and equivalent.

(c) Fatty Acid Glycerides:

The oils which are also suitable are synthetic or semi-synthetic glyceryl esters, such as fatty acid mono-, di or triglycerides, which are modified natural oils or fats, for example glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates, and equivalent.

(d) Non-Volatile Hydrocarbons:

Non-volatile hydrocarbons such as paraffins, isoparaffins, mineral oils, and equivalent are also very suitable for the compositions according to the invention, as non-volatile non-polar solvent.

(e) Guerbet Esters:

Guerbet esters are esters resulting from the reaction of a Guerbet alcohol of general formula: 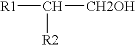
and a carboxylic acid of general formula:
R3-COOH or HOOC—R3-COOH, wherein
R1 and R2, which may be identical or different, are each an alkyl radical having from 4 to 20 carbon atoms, and R3 is a substituted or unsubstituted fatty radical, such as a linear or branched, saturated or unsaturated alkyl or alkylene chain having from 1 to 50 carbon atoms, a phenyl radical, which may be substituted with a halogen, a hydroxyl, a carboxyl, or an alkylcarbonylhydroxyl.

(f) Silicone Oils:

The silicone oils according to the invention for constituting the non-volatile phase are polyorganosiloxane compounds having a measurable pressure under ambient conditions and a viscosity strictly greater than 10 centistokes and lower than 20 centistokes. The non-volatile silicones according to the invention are the compounds of formula: 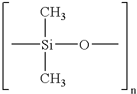
with n strictly greater than 6.

The preferred non-volatile oily phase according to the invention is paraffin oil.

Advantageously, the compositions according to the invention comprise from 1 to 50% by weight, relative to the total weight of the composition, of non-volatile oily phase, preferably from 5 to 30% by weight, and more preferably from 7 to 15% by weight.

According to a preferred embodiment of a composition according to the invention, the composition also comprises a silicone gum. It has, in fact, been discovered, surprisingly, that a composition comprising a silicone gum in the concentrations defined hereinafter exhibit more rapid penetration of the active agent through the various layers of the skin.

The term “silicone gums” means the silicone gums known to those skilled in the art, and in particular those described in EP-0-966,972, incorporated herein by way of reference. According to this preferred embodiment of a composition according to the invention, the silicone gum is introduced at a concentration of from 0.001 to 3% by weight, preferably from 0.01 to 1% by weight. Dow Corning provides a commercial product marketed as DC Silmogen Carrier, which is constituted of 99% of hexamethyldisiloxane and 1% of silicone gum, which product may advantageously be included in one of the compositions according to the invention.

The pharmaceutically acceptable vehicle according to the invention should be selected such that the advantageous properties intrinsically associated with the present invention are not, or are not substantially, altered by the envisaged addition. Preferably, the vehicle used according to the invention is selected so as to be an agent which solubilizes the active agent. The active agent-solubilizing vehicle may be a single excipient, such as a solvent, or of a mixture of excipients, such as those used for the formulation of an emulsion. By way of non-limiting examples of excipients which may be used alone or as a mixture, mention may be made of water, solvents, diluents, and any excipient which can be used for the formulation of an emulsion, of a milk, of a gel, of an ointment, or of a foaming composition. These excipients are compounds commonly used in the formulation of a pharmaceutical composition. Preferably, the active agent-solubilizing excipients according to the invention are water, alcohols, polyols, ethers, esters, aldehydes, ketones, fatty acids and fatty alcohols, and fatty esters. More preferably, the excipient will be an alcohol. According to the invention, the term “alcohol” means linear or branched aliphatic alcohols such as ethanol, propanol or isopropanol.

In a preferred embodiment according to the invention, the vehicle will therefore be alcoholic.

According to the invention, the term “alcoholic vehicle” means a vehicle comprising at least 15% of alcohol, and preferably at least 25% of ethanol.

The pharmaceutical compositions according to the invention may also contain inert additives or combinations of these additives, such as:
wetting agents;
flavor enhancers;
preservatives;
stabilizers;
moisture regulators;
pH regulators;
osmotic pressure modifiers;
emulsifiers;
UV-A and UV-B screening agents;
propenetrating agents;
antioxidants;
and synthetic polymers.

Of course, one skilled in this art will take care to choose the possible compound(s) to be added to these compositions in such manner that the advantageous properties intrinsically associated with the present invention are not, or are not substantially, altered by the envisaged addition.

The compositions according to the invention are more particularly suited for treating the skin and the mucous membranes, and may be provided in the form of ointments, creams, milks, salves, powders, impregnated pads, syndets, solutions, gels, sprays, foams, suspensions, lotions, sticks, shampoos, pledgets or washing bases. They may also be provided in the form of suspensions of lipid or polymer vesicles or nanospheres or microspheres or polymer patches and hydrogels to allow controlled release. This topical-application composition may be provided in anhydrous form, in aqueous form or in the form of an emulsion.

The compositions according to the invention showing improved penetration are preferably administered in the form of a sprayable composition. In order to be sprayable, the compositions according to the invention will preferably have a viscosity of less than 50 centistokes, and more preferably less than 10 centistokes.

The sprayable form, or spray, can be obtained by conventional formulation means known to those skilled in the art. For example, the composition may be sprayed by means of a mechanical spraying device which pumps the composition from a container, bottle or equivalent. The composition passes through a nozzle which can be aimed directly at the desired site of application. The nozzle can be selected so as to apply the composition in the form of a vaporization or of a jet of droplets, according to techniques known to those skilled in the art. According to the pharmaceutical active agent selected, the spraying mechanism must be capable of always delivering the same amount of active agent. The mechanisms for controlling the amount of composition to be delivered by the spray are also known to those skilled in this art.

Preferably, for the compositions according to the invention, a dosing spray bottle, for which the application area and dose characteristics are controlled and reproducible, will be used. For example, the spray device may be a bottle equipped with a 25 μl dosing valve.

The present invention also features the use of a composition according to the invention, for producing a medicinal product suited for treating (whether regime or regimen):

dermatological conditions or afflictions associated with a keratinization disorder relating to differentiation and to proliferation, in particular common acne, comedo-type acne, polymorphic acne, rosacea, nodulocystic acne, acne conglobata, senile acne, and secondary acne such as solar, drug-related or occupational acne,

ichthyoses, ichthyosiform conditions, Darrier's disease, palmoplantar keratoderma, leukoplakia and leukoplakiform conditions, and cutaneous or mucosal (oral) lichen,

dermatological conditions or afflictions with an inflammatory immunoallergic component, with or without a cell proliferation disorder, in particular cutaneous, mucosal or ungual psoriasis, psoriatic rheumatism, cutaneous atopy, such as eczema, respiratory atopy or gingival hypertrophy,

benign or malignant dermal or epidermal proliferations, of viral or non-viral origin, in particular common warts, flat warts, epidermodysplasia verruciformis, oral or florid papillomatoses, and T lymphoma,

proliferations which may be induced by ultraviolet light, in particular basal cell epithelioma and spinocellular epithelioma,

precancerous skin lesions, in particular keratoacanthomas,

immune dermatoses, in particular lupus erythematous,

bullous immune diseases,

collagen diseases, in particular scleroderma,

dermatological or systemic conditions or afflictions with an immunological component,

skin disorders due to exposure to UV radiation, or light-induced or chronological aging of the skin, or actinic keratoses and pigmentations, or any pathologies associated with chronological or actinic aging, in particular xerosis,

sebaceous function disorders, in particular hyperseborrhoea acne or simple seborrhoea or seborrhoeic dermatitis,

cicatrization disorders or stretch marks,

pigmentation disorders, such as hyperpigmentation, melasma, hypopigmentation or vitiligo,

lipid metabolism conditions, such as obesity, hyperlipidemia, non-insulin-dependant diabetes or syndrome X,

inflammatory conditions or afflictions such as arthritis,

cancerous or precancerous states,

alopecia of various origins, in particular alopecia caused by chemotherapy or radiation,

immune system disorders, such as asthma, type 1 diabetes mellitus, multiple sclerosis or other selective dysfunctions of the immune system, or

cardiovascular system conditions such as arteriosclerosis or hypertension.

In a preferred embodiment of use of the composition, said composition will contain 0.3% of (4E,6E)-7-[3-(3,4-bishydroxymethylbenzyloxy)phenyl]-3-ethylnona-4,6-dien-3-ol and will be used for formulating a medicinal product suited to treat psoriasis.

This invention also features a process for improving the penetration of an active agent derived from vitamin D, wherein a composition comprising the following, in a pharmaceutically acceptable vehicle, is topically applied onto the skin:
a) a therapeutically effective amount of a bioactive agent derived from vitamin D,
b) at least one volatile silicone,
c) a non-volatile oily phase,
   said composition being applied in the form of a spray.

Indeed, it has now been discovered, surprisingly, that the penetration of an active agent, and in particular of compounds derived from vitamin D, through the skin is improved by the composition according to the invention, even in the absence of excipients with propenetrating activity.

The expression “improvement in penetration into the skin” means a significant increase in penetration into the skin of at least a factor of 2, compared to formulations previously produced on the market.

The penetration of the active agent is measured according to the protocol described in Example 4 to follow.

The following examples illustrate formulation of the compositions according to the invention and results of penetration into the skin.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

The formulation is obtained by mixing the various compounds indicated below until a homogeneous and clear solution is obtained.

| Ingredients | Function | Spray A |
|---|---|---|
| (4E,6E)-7-[3-(3,4- | Active agent | 0.3% |
| bishydroxymethylbenzyloxy)phenyl]- | | |
| 3-ethylnona-4,6-dien-3-ol | | |
| Hexamethyldisiloxane | Volatile silicone | 60.0% |
| Paraffin oil | Non-volatile oily | 10.0% |
| Absolute ethanol | phase | qs 100% |
| | Solvent: excipient | |

### EXAMPLE 2

The procedure is the same as that in Example 1.

| Ingredients | Function | Spray B |
|---|---|---|
| (4E,6E)-7-[3-(3,4- | Active agent | 0.3% |
| bishydroxymethylbenzyloxy)phenyl]- | | |
| 3-ethylnona-4,6-dien-3-ol | | |
| Hexamethyldisiloxane | Volatile silicone | 59.4% |
| Silicone gum | Silicone gum | 0.6% |
| Paraffin oil | Non-volatile oily | 10.0% |
| Absolute ethanol | phase | qs 100% |
| | Solvent | |

### EXAMPLE 3

The procedure is the same as that in Example 1.

| Ingredients | Function | Spray C |
|---|---|---|
| (4E,6E)-7-[3-(3,4- | Active agent | 0.3% |
| bishydroxymethylbenzyloxy)phenyl]- | | |
| 3-ethylnona-4,6-dien-3-ol | | |
| Hexamethyldisiloxane | Volatile silicone | 59.4% |
| Silicone gum | Silicone gum | 0.6% |
| Paraffin oil | Non-volatile oily | 10.0% |
| Oleic acid | phase | 5.0% |
| Butylhydroxytoluene | Propenetrating | 0.05% |
| (BHT) | agent | |
| Absolute ethanol | Antioxidant | qs 100% |
| | Solvent | |

### EXAMPLE 4

Study of the release/penetration in vitro, on human skin, of (4E,6E)-7-[3-(3,4-bishydroxymethylbenzyloxy)phenyl]-3-ethylnona-4,6-dien-3-ol contained in two different formulations, one of which is sprayable according to the preferred embodiment of the invention.

The first objective is to quantify the penetration into the skin of the active agent formulated in both formulations, in vitro, on human skin, after 16 hours of application. A sprayable formula according to the invention is compared with a composition in the form of an ointment.

The exact compositions of the two formulations are reported in Table 1 below.

TABLE 1

| | | Spray | Ointment |
|---|---|---|---|
| Ingredients | Function | (in %) | (in %) |
| (4E,6E)-7-[3-(3,4- | Active agent | 0.3 | 0.3 |
| bishydroxymethyl- | | | |
| benzyloxy)phenyl]- | | | |
| 3-ethylnona-4,6- | | | |
| dien-3-ol | | | |
| Hexamethyldisiloxane | Volatile silicone | 59.40 | |
| Silicone gum | Silicone gum | 0.6 | |
| Paraffin oil | Occlusive Agent | 10.00 | 5 |
| Oleic acid | Propentrating agent | / | / |
| Propylene glycol | Propentrating agent | | 10.00 |
| White petroleum jelly | Occlusive agent | | 76.94 |
| Macrogol 2 stearyl ether | | | 5 |
| Disodium EDTA | Chelating agent | | 0.0065 |
| DL-alpha-tocopherol | Antioxidant | | 0.12 |
| BHT | Antioxidant | | |
| Absolute ethanol | Solvent | qs 100% | |
| Water | Solvent | | qs 100% |

Experimental Conditions:

Percutaneous absorption is evaluated by means of diffusion cells consisting of 2 compartments separated by human skin. The formulations were applied without occlusion for 16 hours. The formulations were applied at a rate of 10 mg of formulation per cm² (i.e., 30 micrograms of (4E,6E)-7-[3-(3,4-bishydroxymethylbenzyloxy)phenyl]-3-ethylnona-4,6-dien-3-ol). Throughout the duration of the study, the dermis is in contact with a recipient liquid which is not renewed as a function of time (static mode). At the end of the application period, the surface excess is removed and the distribution of the (4E,6E)-7-[3-(3,4-bishydroxymethylbenzyloxy)phenyl]-3-ethylnona-4,6-dien-3-ol is quantified in the various skin compartments and in the recipient liquid. The concentrations of (4E,6E)-7-[3-(3,4-bishydroxymethylbenzyloxy)phenyl]-3-ethylnona-4,6-dien-3-ol were quantified using an HPLC/MS/MS method conventionally known to those skilled in the art (LQ: 10 ng.mL⁻¹). The spray formula was applied using a spray bottle equipped with a 25 μl dosing valve.

The experimental results show that, whichever the formulation tested, the active agent is distributed mainly in the skin (epidermis, including strateum corneum, and dermis). The total amounts penetrated (stratum corneum+epidermis+dermis+recipient liquid) are:

| | Application time: 16 hours |
|---|---|
| Spray | |
| Total amount having penetrated | |
| μg | 2.64 ± 0.50 μg |
| % dose applied | 9.2% |
| Ointment | |
| Total amount having penetrated | |
| μg | 0.63 ± 0.14 μg |
| % dose applied | 2.3% |

Results:

The results here demonstrate that the spray formula according to the invention shows a four-fold increase in penetration of the bioactive agent after 16 hours, compared with the ointment formula.

This result therefore indicates that the compositions according to the invention make it possible to obtain a significant improvement in penetration of an active agent derived from vitamin D compared with existing formulas.

The spray formulas as described therefore make it possible to avoid the use of glycols, without decreasing skin penetration, and therefore show an additional advantage in terms of nonirritant potential versus the compositions comprising a high content of glycol.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A sprayable pharmaceutical composition suited for enhanced transdermal penetration of at least one bioactive agent, comprising a) said at least one bioactive agent, (b) at least one volatile silicone and c) a non-volatile oily phase, formulated into d) a pharmaceutically acceptable vehicle therefor.

2. The sprayable pharmaceutical composition as defined by claim 1, said at least one bioactive agent a) comprising a derivative of vitamin D.

3. The sprayable pharmaceutical composition as defined by claim 1, said at least one bioactive agent a) comprising a derivative of vitamin D₂ or D₃, or comprising calcitriol.

4. The sprayable pharmaceutical composition as defined by claim 2, said at least one bioactive agent a) being selected from the group consisting of:
6-[3-(3,4-bishydroxymethylbenzyloxy)phenyl]-2-methylhepta-3,5-dien-2-ol,
7-[3-(3,4-bishydroxymethylphenoxymethyl)phenyl]-3-ethyloctan-3-ol,
7-{3-[2-(3,4-bishydroxymethylphenyl)ethyl]phenyl}-3-ethylocta-4,6-dien-3-ol,
6-{3-[2-(3,4-bishydroxymethylphenyl)ethyl]phenyl}-2-methylhepta-3,5-dien-2-ol,
7-{3-[2-(3,4-bishydroxymethylphenyl)vinyl]phenyl}-3-ethylocta-4,6-dien-3-ol,
7-[3-(3,4-bishydroxymethylbenzyloxy)phenyl]-3-ethyl-3-octanol,
4E,6E)-7-[3-(3,4-bishydroxymethylbenzyloxy)phenyl]-3-ethylocta-4,6-dien-3-ol,
(4E,6E)-7-[3-(3,4-bishydroxymethylbenzyloxy)phenyl]-3-ethylnona-4,6-dien-3-ol,
(E)-7-[3-(3,4-bishydroxymethylbenzyloxy)phenyl]-3-ethyloct-4-en-3-ol,
(E)-7-[3-(3,4-bishydroxymethylbenzyloxy)phenyl]-3-ethyloct-6-en-3-ol,
(E)-7-[3-(3,4-bishydroxymethylbenzyloxy)phenyl]-3-ethyloct-6-en-4-yn-3-ol,
(4E,6E)-7-[3-(3,4-bishydroxymethylphenoxymethyl)phenyl]-3-ethylocta-4,6-dien-3-ol,
(E)-7-[3-(3,4-bishydroxymethylphenoxymethyl)phenyl]-3-ethylnon-6-en-3-ol,
(E)-7-{3-[(3,4-bishydroxymethylbenzyl)methylamino]phenyl}-3-ethyloct-6-en-3-ol, and
7-[3-(3,4-bishydroxymethylbenzyloxy)phenyl]-3-ethyl-7-methyloctan-3-ol.

5. The sprayable pharmaceutical composition as defined by claim 1, comprising:
a) from 0.0001 to 20% by weight of said at least one bioactive agent,
b) from 25 to 95% by weight of said volatile silicone,
c) from 1 to 50% by weight of said non-volatile oily phase.

6. The sprayable pharmaceutical composition as defined by claim 4, said at least one bioactive agent a) comprising (4E,6E)-7-[3-(3,4-bishydroxymethylbenzyloxy)phenyl]-3-ethylnona-4,6-dien-3-ol.

7. The sprayable pharmaceutical composition as defined by claim 2, comprising from 0.01% to 2% by weight of said derivative of vitamin D.

8. The sprayable pharmaceutical composition as defined by claim 1, said pharmaceutical acceptable vehicle comprising an alcoholic vehicle.

9. The sprayable pharmaceutical composition as defined by claim 8, said alcoholic vehicle comprising at least 15% of alcohol.

10. The sprayable pharmaceutical composition as defined by claim 9, said alcoholic vehicle comprising at least 25% of ethanol.

11. The sprayable pharmaceutical composition as defined by claim 1, said at least one volatile silicone being selected from the group consisting of polydimethylcyclosiloxanes and low molecular weight linear polysiloxanes.

12. The sprayable pharmaceutical composition as defined by claim 11, said at least one volatile silicone comprising a linear polysiloxane of hexamethyldisiloxane type.

13. The sprayable pharmaceutical composition as defined by claim 12, comprising from 55 to 65% by weight of hexamethyldisiloxane.

14. The sprayable pharmaceutical composition as defined by claim 1, said non-volatile oily phase comprising a non-polar oil.

15. The sprayable pharmaceutical composition as defined by claim 14, said non-polar oil comprising paraffin oil.

16. The sprayable pharmaceutical composition as defined by claim 15, comprising from 5 to 15% of paraffin oil.

17. The sprayable pharmaceutical composition as defined by claim 1, further comprising a silicone gum.

18. The sprayable pharmaceutical composition as defined by claim 1, comprising:
a) 0.3% of (4E,6E)-7-[3-(3,4-bishydroxymethylbenzyloxy)phenyl]-3-ethyinona-4,6-dien-3-ol,
b) 60% of hexamethyldisiloxane,
c) 10% of paraffin oil,
d) 29.7% of ethanol.

19. The sprayable pharmaceutical composition as defined by claim 1, comprising:
a) 0.3% of (4E,6E)-7-[3-(3,4-bishydroxymethylbenzyloxy)phenyl]-3-ethylnona-4,6-dien-3-ol,
b) 59.4% of hexamethyldisiloxane,
c) 0.6% of silicone gum,
d) 10% of paraffin oil,
e) 29.7% of ethanol.

20. A regime or regimen for the treatment of:
dermatological conditions or afflictions associated with a keratinization disorder relating to cell differentiation and proliferation;
ichthyosis, ichthyosiform conditions, Darier's disease, palmoplantar keratoderma, leukoplakia and leukoplakiform conditions, and cutaneous or mucous (buccal) lichen;
dermatological conditions or afflictions having an inflammatory immunoallergic component, with or without a cell proliferation disorder;
benign or malignant dermal or epidermal proliferations, of viral or non-viral origin;
proliferations that may be induced by ultraviolet radiation;
precancerous skin lesions;
immune dermatoses;
immune bullous diseases;
collagen diseases;
dermatological conditions or afflictions having an immunological component;
stigmata of epidermal and/or dermal atrophy induced by local or systemic corticosteroids, or any other form of cutaneous atrophy;
skin conditions or afflictions of viral origin;
skin disorders caused by exposure to UV radiation, photoinduced or chronological aging of the skin, or actinic pigmentations and keratoses;
pathologies associated with chronological or actinic aging of the skin;
disorders of sebaceous function;
cicatrization disorders or stretch marks; or
pigmentation disorders;
lipid metabolism conditions or disorders;
inflammatory conditions or disorders;
immune system disorders;
alopecia;
cardiovascular system conditions or disorders; comprising administering to a mammalian organism in need of such treatment, a thus effective amount of a compound as defined by claim 1.

21. The regime or regimen as defined by claim 20, comprising the treatment of psoriasis.

22. A process for improving the transdermal penetration of at least one pharmaceutical bioactive agent comprising a derivative of vitamin D, which comprises spraying onto the skin:
a) a therapeutically effective amount of at least one derivative of vitamin D,
b) at least one volatile silicone,
c) a non-volatile oily phase,
d) formulated into a pharmaceutically acceptable vehicle therefor.

23. The process as defined by claim 22, said at least one derivative of vitamin D comprising (4E,6E)-7-[3-(3,4-bishydroxymethylbenzyloxy)phenyl]-3-ethylnona-4,6-dien-3-ol, the volatile silicone comprising hexamethyldisiloxane, and also comprising paraffin oil.

24. The process as defined by claim 22, also comprising spraying a silicone gum onto the skin.

25. The sprayable pharmaceutical composition as defined by claim 2, said at least one derivative of vitamin D having the following structural formula (I): 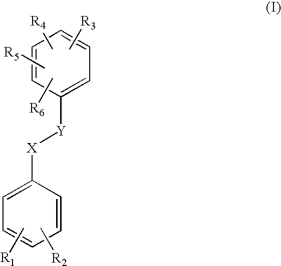
in which:
R₁ is a hydrogen atom, a methyl radical or a radical —(CH₂)ₙ—OR₇;
R₂ is a radical —(CH₂)ₙ—OR₈;
n, R₇ and R₈ are as defined below;
X—Y is a bond selected from the bonds of formulae (a) to (d) below which can be read from left to right or vice versa: 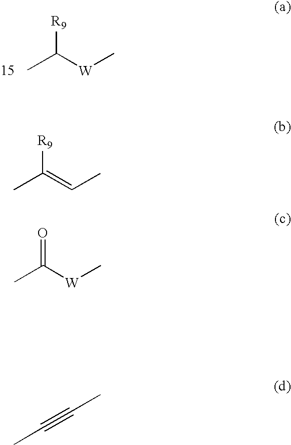
R₉ and W having the meanings given below;
R₃ is the chain of vitamin D₂ or vitamin D₃; 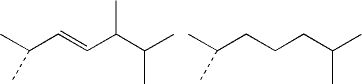
the dashed lines represent the bond linking the chain to the benzene ring represented in formula (1),
or R₃ is a chain having from 4 to 8 carbon atoms substituted with one or more hydroxyl groups, with the proviso that the hydroxyl groups may be protected in acetoxy, methoxy or ethoxy, trimethylsilyloxy, tert-butyldimethylsilyloxy or tetrahydropyranyloxy form, and optionally, in addition:
substituted with one or more lower alkyl or cycloalkyl groups and/or
substituted with one or more halogen atoms and/or
substituted with one or more groups CF₃ and/or
in which one or more carbon atoms of the chain are replaced with one or more oxygen, sulfur or nitrogen atoms, with the proviso that the nitrogen atoms may be optionally substituted with lower alkyl radicals and/or
in which one or more single bonds of the chain are replaced with one or more double and/or triple bonds,
R₃ being positioned, on the benzene ring, in the position para- or meta- to the X—Y bond;
R₄, R₅ and R₆, two of which may be identical or different, are each a hydrogen atom, a lower alkyl radical, a halogen atom, a radical —OR₁₀ or a polyether radical;
R₁₀ is as defined below;
n is 0, 1 or 2;
R₇ and R₈, which may be identical or different, are each a hydrogen atom, an acetyl radical, a trimethylsilyl radical, a tert-butyldimethylsilyl radical or a tetrahydropyranyl radical;
R₉ is a hydrogen atom or a lower alkyl radical;
W is an oxygen or sulfur atom, a radical —CH₂— or a radical —NH— which can optionally be substituted with a lower alkyl radical;
R₁₀ is a hydrogen atom or a lower alkyl radical;
and also the optical and geometrical isomers of said compounds of formula (I), and the salts thereof when X—Y are each a bond of formula (a) and W is a radical —NH— optionally substituted with a lower alkyl radical.

26. A dosing spray receptacle comprising a dosing valve and confining the sprayable pharmaceutical composition as defined by claim 1.
